(19) United States
(12) Patent Application Publication
(10) Pub. No.: US 2025/0201100 A1
(43) Pub. Date: Jun. 19, 2025

(54) CONTACT SENSING SYSTEM AND METHOD OF USE THEREOF

(71) Applicant: Tomer TAL, Verdi, NV (US)

(72) Inventor: Tomer TAL, Verdi, NV (US)

(73) Assignee: Tomer TAL, Verdi, NV (US)

(21) Appl. No.: 18/980,679

(22) Filed: Dec. 13, 2024

Related U.S. Application Data
(60) Provisional application No. 63/609,656, filed on Dec. 13, 2023.

(51) Int. Cl. G08B 21/18

(52) U.S. Cl. CPC G08B 21/182

(57) ABSTRACT

Systems and methods for capturing and transmitting human contact and input of a rider with another object, such as a motorcycle, bicycle or horse, to which the rider interacts.

### CROSS-REFERENCED TO RELATED PATENT APPLICATIONS

The application is a non-provisional application of and claims priority benefits to U.S. Patent Appl. Ser. No. 63/609,656, entitled “Contact Sensing System And Method Of Use Thereof,” filed Dec. 13, 2023, to Tomer Tal. This patent application is commonly owned by the Inventor of the present invention and is hereby incorporated herein by reference in its entirety for all purposes.

### TECHNICAL FIELD

The present invention relates to systems and methods for capturing and transmitting human contact and input of a rider with another object, such as a motorcycle, bicycle or horse, to which the rider interacts.

### BACKGROUND

Motorcycles (like other moveable objects that are ridden) perform best when designed with mass centralization and a lower center of gravity. It is the job of the manufacturer to deliver a competitive motorcycle that provides these elements. However, if not ridden properly, the mass centralization and center of gravity of a motorcycle may change dramatically and adversely given the element of rider weight. A typical off-road motorcycle is around 230 pounds. The average American male weights between 195-200 pounds, which is nearly the weight of the motorcycle. Therefore, how and where that weight is distributed on the motorcycle will affect its handling and performance characteristics. By using correct riding technique, a rider can (i) position the center of gravity of the motorcycle in the manner most beneficial for the situation the rider is facing at the time (ii) conserve energy and (iii) reduce the risk of injury. Additionally, the device also has value for road racing motorcycles when frame or peg mounted.

Real time data acquisition and telemetry is well known in many fields, in this case in automotive and motorcycle racing and training. However, to date, such telemetry is (i) very expensive and used by professional teams and (ii) generally limited to suspension and motor functions. Thus, a need remains in the art for an approved system and methods for improving riding of these movable objects, as well as improved systems and methods for training proper riding.

### SUMMARY OF THE INVENTION

The present invention relates to systems and methods for capturing and transmitting human contact and input of a rider with another object, such as a motorcycle, bicycle or horse, to which the rider interacts.

The present invention relates to a data logging and transmission device that measures rider position data, such as a dirt bike rider on the motorcycle (or, for example, an equestrian on a horse) with respect to (i) amount of time sent sitting on a motorcycle seat (in this case a dirt bike seat) vs. standing on the pegs and off the seat, (ii) the amount of time (and possibly amount of pressure) a rider spends gripping the motorcycle frame with the rider's legs and or ankles vs riding loose and away from the bike and (iii) the number of times or amount of time a riders boot leaves the foot peg in order to dab, paddle or otherwise use the leg as a pivot point. All this information can be utilized to correct poor and or unsafe technique in real time through auditory, tactile, visual, or other sensation (such as by a buzzing sound, by vibration, or by a strobe light) on one or more wearable units (such as an armband) as well as stored and recorded for later review.

In general, in another embodiment, the invention features a system for motion tracking, capturing, and transmitting a rider contact and input with a movable object. The system includes one or more pressure sensors that are each removably attachable to the movable object or rider such that the one or more pressure sensor can be placed at one or more locations of the movable object or rider. The one or more pressure sensors are located on the movable object or rider to detect the presence and amount of force applied by the rider to the movable object at the one or more locations. The system further includes a tracker removably attachable to the movable object or the rider. The tracker is capable of tracking where the movable object is. The system further includes a recorder that is operably connected to the one or more pressure devices and the tracker. The recorder is operable to record information that includes (i) the presence and amount of force applied by the rider to the movable object at the one or more locations, and (ii) where the movable object is at the time when the presence and amount of force applied by the rider to the movable object at the one or more locations. The system further includes an indicator that is operable to provide real-time feedback to the rider related to the information recorded by the recorder. The indictor is removably attachable to the movable object or the rider.

Implementations of the invention can include one or more of the following features:

The one or more pressure sensors can be wirelessly connected to recorder.

The one or more pressure sensors can be coupled to a blue-tooth transmitter for wireless communication with the recorder.

The recorder can include a CPU.

The CPU can be operable to record times of contact and times of non-contact of the one or more pressure devices.

The CPU can be programmed to read, buffer, store, and filter information from the one or more pressure sensors.

The tracker can include a GPS system.

The CPU can be operable to compare locations obtained using the GPS system. The CPU can be further operable to process the information from the one or more pressure sensors and the GPS.

The CPU can be operable to transmit the processed information to a mobile device.

The CPU can be operable to transmit the information from the one or more pressure sensors and the GPS to a mobile device.

The tracker can include a GPS system.

The tracker can include an accelerometer.

The accelerometer can track tilt of the movable object.

The system can include a combination of hardware and software. The hardware can include a GPS receiver, a CPU, and a wireless transmitter. The software can include an app or web application that can receive and process data from the CPU and the GPS receiver. The app or web application can be operable to chart and display the rider's activities along with a map chart of where the activity occurred based on the GPS data. The app or web application can be operable to store the data for future use.

Each data streams recorded by the recorder from each of the one or more pressure sensors can be displayed individually or collectively with other information recorded by the recorder, in a rider chosen variety of colors to distinguish each data stream.

The recorder can be operable for downloading information recorded by the recorder to a storage device that is not attached to the movable object or the rider.

The recorder can further include a blue-tooth transmitter for wireless communication with the storage device.

The movable object can be a vehicle.

The vehicle can be a non-motorized cycle.

The vehicle can be a motorized cycle.

The movable object can be an animal.

The animal can be a horse.

At least one of the one more pressure sensors can be removably attachable to a part of the movable object selected from the group consisting of a seat, saddle, bicycle peddle, motorcycle peg, horse stirrup, and handlebar of the movable object.

Each of the one or more pressure sensors can include a flexible adhesive operable for removably attaching each of the one or more pressure sensors to the movable object or the rider.

The tracker can include a flexible adhesive operable for removably attaching the device to the movable object.

The recorder can include a flexible adhesive operable for removably attaching the device to the movable object.

The tracker can be housed in a small puck that protects the tracker.

The recorder can be housed in a small puck that protects the recorder.

The tracker and recorder can be housed together.

The tracker and recorder can be housed together in a small puck that protects the tracker and recorder.

The indicator can provide feedback to the rider that is visual response.

The visual response can be provided only when the movable object is stopped.

The visual response can be a flashing light.

The indicator can be a strobe light.

The indicator can provide feedback to the rider that is an audible response.

The audible response can be a buzzing sound.

The indicator can be a buzzer.

The system can provide feedback to the rider that is a tactile response.

The tactile response can be a vibration.

The indicator can be a vibrator.

The indictor can be removably attachable to the rider.

The indicator can include a plurality of wearable indicators.

The plurality of wearable indicators can include a right-side wearable indicator and a left-side wearable indicator.

The right-side wearable indicator can be removably attachable to a right wrist or a right arm of the rider. The left-side wearable indicator can be removably attachable to a left wrist or a left arm of the rider.

The right-side wearable indicator and the left-side wearable indicator can be independently controllable.

The system can be operable to selectively provide real-time feedback to the rider on some, but not all, of the wearable indicators in the plurality of wearable indicators.

The can be operable to selectively provide real-time feed to the rider of only one of the wearable indicators in the plurality of wearable indicators.

The one or more pressure sensor can include a first pressure sensor, a second pressure sensor, and a third pressure sensor. The first pressure sensor can be removable attachable at a first location that is at a right knee rest on the movable object or at or by a right knee of the rider. The second pressure sensor can be removable attachable at a second location that is at a left knee rest on the moveable object or at or by a left knee of the rider. The third pressure sensor can be removable attachable to a seat of the movable object. The indicator can include a first wearable indicator and a second wearable indicator. The first wearable indicator can be removably attachable to a right wrist or a right arm of the rider. The second wearable indicator can be removably attachable to a left wrist or a left arm of the rider.

The system can be operable in real time to provide a first set of indications to the rider, via the first wearable indicator, based upon information recorded from the recorder obtained from the first pressure sensor. The system can be operable in real time to provide a second set of indications to the rider, via the second wearable indicator, based upon information recorded from the recorder obtained from the second pressure sensor. The system can be operable in real time to provide a third set of indications to the rider, simultaneously via the first wearable indicator and the second wearable indicator, based upon information recorded from the recorder obtained from the third pressure sensor.

In general, in another embodiment, the invention features a method of using any of the above-described systems.

In general, in another embodiment, the invention features a method for motion tracking, capturing, and transmitting a rider contact and input with a movable object. The method includes selecting a system that includes one or more pressure sensors that are each removably attachable to the movable object or rider. The system further includes a tracker removably attachable to the movable object or the rider. The system further includes a recorder that is operably connected to the one or more pressure devices and the tracker. The system further includes an indicator removably attachable to the movable object or the rider. The method further includes attaching the one or more pressure sensors to the movable object or rider such that the one or more pressure sensor are placed at one or more locations of the movable object or rider. The method further includes attaching the tracker to the movable object or the rider. The method further includes utilizing the system when the rider is riding the movable object. Utilizing the system includes the one or more pressure sensors detect the presence and amount of force applied by the rider to the movable object at the one or more locations. Utilizing the system further includes the tracker tracks where the movable object is. Utilizing the system further includes the recorder records information that includes (A) the presence and amount of force applied by the rider to the movable object at the one or more locations, and (B) where the movable object is at the time when the presence and amount of force applied by the rider to the movable object at the one or more locations. Utilizing the system further includes the indicator provides real-time feedback to the rider related to the information recorded by the recorder.

Implementations of the invention can include one or more of the following features:

The method can improve the rider's riding of the movable object.

The method can be a used to train the rider to ride the moveable object.

The method can include the rider riding a movable object with each of the one or more pressure sensors, tracker, recorder, and indicator of the system attached to the movable object and/or rider. The method can further include the rider receives real-time feedback from the indicator while riding the movable object.

The rider can adjust the rider's riding of the movable object in response to the real-time feedback from the indicator.

When the rider is riding the movable object outside pre-set parameters for the position of a right knee of the rider relative to a right knee rest of the movable object, the first wearable indicator can provide an indication to the rider. When the rider is riding the movable object outside pre-set parameters for the position of the left knee of the rider relative to a left knee rest of the movable object, the second wearable indicator can provide an indication to the rider. When the rider is riding the movable object outside pre-set parameters for the position of the rider relative to seat of the movable object, the first wearable indicator and the second wearable indictor can simultaneously provide an indication to the rider.

### BRIEF DESCRIPTION OF THE DRAWINGS

FIGS. 1A-1C are illustrations showing a system of the present invention.

FIG. 2 illustrates the transmission of information using the system that can be used in the embodiment shown in FIGS. 1A-1C.

FIG. 3 shows a block diagram for the sensor array for the seat and knee rest that can be used in the embodiment shown in FIG. 1A.

FIG. 4 shows a block diagram for vibrating bracelet that can be used in the embodiment shown in FIG. 1B.

FIG. 5A is an illustration of a pressure sensor that can be used in the embodiment shown in FIG. 1A.

FIG. 5B is an illustration of a flexible pressure sensor array that can be used in the embodiment shown in FIG. 1A.

FIG. 5C is an illustration of an exploded view of the flexible pressure sensor array shown in FIG. 5B.

FIG. 6 is an illustration of a vibrating bracelet that can be used in the embodiment shown in FIG. 1B.

FIG. 7 is an illustration of a mobile device display that can be used in the embodiment shown in FIG. 1C.

FIGS. 8A-8C are illustrations of seat pressure sensor for further embodiments of the invention. FIG. 8A shows a motorcycle having a seat pressure sensor. FIG. 8B an overhead view of the seat (including the seat pressure sensor) of the motorcycle shown in FIG. 8A. FIG. 8C an underside view of the seat (including the seat pressure sensor) of the motorcycle shown in FIG. 8A.

FIGS. 9A-9B are illustrations of a knee sensor for further embodiments of the invention. FIG. 9A shows a flexible sensor array of pressure sensors that can be attached to a knee of the rider. FIG. 9B shows the flexible sensor array of FIG. 9A attached to a knee of the rider.

### DETAILED DESCRIPTION

The present invention present invention relates to systems and methods for capturing and transmitting human contact and input of a rider with another object, such as a motorcycle, bicycle or horse, to which the rider interacts.

In embodiments, the system and method includes a device for capturing and transmitting human contact and input with another object such a motorcycle, bicycle or horse. The device would capture and transmit human movement through contact points on the object it was attached to, for example, a seat, or saddle, bicycle peddle or motorcycle peg or horse stirrup, or the side of a motorcycle or bicycle. While the discussion below is directed to cycles (such as off-road motorcycles, referred to as dirt-bikes), the present invention applies to other types of cycles (such as motocross, enduro or other forms of off-road motorcycle racing) and other types of movable objects, even horses (such as for equestrians on horses).

When using the present invention, a rider can be able to know when gripping the vehicle or other object (such as the side of dirt bike) and when the rider is not gripping the vehicle or other object. For instance, a pressure sensor can detect and convey the intensity of pressure generated by a human against the sensor and convey that data to a CPU that would then buffer, store, filter, and then broadcast that information, potentially with accompanying GPS coordinates, via blue-tooth or other signal, to an application housed on the phone or the cloud as well as providing real-time feedback to the user through auditory, tactile, visual, or other sensation (such as by a buzzing sound, by vibration, or by a strobe light) on one or more wearable units (such as an armband).

The present invention provides a device for improved riding and training that includes (i) a pressure (or force) sensor that senses body weight or pressure against it (ii) a datalogger that records when the pressure sensor was activated as well as length of activation and (iii) a transmitter that transmits the data, such as via Bluetooth, to a phone or watch. Alternatively, the transmission could consist of an audible sound or a tactile response (such as vibration).

In embodiments, using an app, the rider can overlay GPS data against mapping software or perhaps a performance service (such as Strava).

On a motorcycle, the device can be attached to the motorcycle seat (to determine seated vs standing time), the frame rails (to determine leg grip both in terms of time and alternatively/additionally amount of pressure), and on the pegs (to determine when the rider's foot is off the peg and therefore affecting the balance of the motorcycle).

The device can utilize a pressure sensor on top of the seat (or underneath the seat cover) encased in a rubber strip, ace bandage, or any other flexible fabric. Force sensors for this sort of application are available commercially with a thickness of less than one millimeter. An array of these sensors could be sewn into a flexible fabric along with (i) a power source, such as rechargeable via USB cable (ii) a blue-tooth transmitter (iii) a computer chip (custom ARM) that records and monitors the triggering of the force sensor, buffers the data and filters and processes the data prior to transmitting it, via blue-tooth to a cellular phone (or watch). The GPS functionality and mapping features etc. can be offloaded to a phone where the processing power resides. In addition, because the GPS data can be housed on a phone, the app can be able to cease measuring data if it detects a lack of movement. For example, a rider stopped at trailhead while still seated.

Alternatively or additionally, the system of the present invention can be integrated into a wearable, for example riding pants in the seat and a vertical strip down each leg with multiple contact points. By measuring and mapping the force, one could also ascertain any bi-lateral imbalances in strength, which may be indicative of nerve damage/compression or other potential health related issues.

### Pressure Sensor

The pressure sensor is operable to measure the presence and/or non-presence of the rider's weight, as well as the amount of force being applied against the sensor. This data can then be transmitted to a computer or other device for further processing.

The pressure sensor is a device that can measure the amount of pressure applied to it. In some embodiments, it is can be made up of a thin, flexible material that is sensitive to pressure. This device is then transmitted, via electronic signal to a customer computing device located on the object. The pressure sensor is able to detect the amount of pressure applied to it and send a signal to the computer or other device.

The pressure sensor can be placed in various places on a device. This could include the handle of a device, the buttons on a device, or the surface of a device. The pressure sensor is operable to detect the amount of pressure applied to it and send a signal to the computer or other device. This signal is then used to track the force placed against the device along with a GPS tracker in the CPU showing the location where the force was applied.

### Force Tracking

The force tracking process can include the pressure sensor detecting the amount of pressure applied to it. This information is then sent to the computer or other device, which then records the presence, non-presence and/or amount of force applied against the pressure sensor. This data is then used to track the riding behavior of the party applying the force. The user can determine the amount of force necessary to trigger the presence or absence of force by the sensor.

### Motion Tracking

The motion tracking process is a novel way of capturing and transmitting human contact and input. By using a pressure sensor, the device is able to record the presence, non-presence and/or amount of force applied against the pressure sensor. This data is then used to track the rider behavior, allowing for a variety of applications such as safety training as well as possibly ascertaining if there are any injuries, nerve damage or other limitations that preclude a rider from using the optimal and safest technique.

The device is placed in the environment where motion tracking is desired. The device can be equipped with sensors that detect contact and non-contact. When the device senses the absence of contact, it can send a signal to the CPU. The CPU then can record the time of contact and time of non-contact.

### Components of Device

The device is designed to and can capture and transmit data. It can be equipped with a microprocessor that is programmed to detect contact and non-contact. The microprocessor is also programmed to send a signal to the CPU when it senses the absence of contact. This signal is then used to record the times of contact and non-contact.

The device can also have a built-in memory, which stores the data collected by the device.

The device can also be equipped with a wireless transmitter that transmits the data to the microprocessor (CPU) potentially along with GPS information to allow a user to map and chart the area (trail, track, etc.) where the user was operating the device.

### Operation

The data collected by the device can be used to create a tracking map. This map can be used to visualize the movements of the object or person being tracked. The motion-tracking map can be used to analyze the movements of the object or person and can be used to make decisions about future actions.

Utilizing this device, the pressure and tracking can be used to record and measure the movement of an object and its rider (and how they interact). The device captures and transmits data by sending a signal to the CPU when it senses the either the presence of, or the absence of contact, and wherein the CPU records the times of contact and times of non-contact implemented by said device. This process can be used to create a motion tracking map which can be used to analyze the movements of the object or person and make decisions about future actions.

### Cycling Device

Representative of the present invention is a device used by a cyclist or motorcyclist. Such a cyclist can use the device and is able to select from a variety of locations on the cycle so that that the rider can ride safely and securely it (and also position it to learn that they are riding the cycle correctly (such as to maintain proper posture on the cycle when operation).

By way of example, a cyclist can elect on the proper positions to attach the device to the cycle. I.e., the rider can attaching the device to the seat, saddle, bicycle peddle, motorcycle peg, horse stirrup, or handlebars, etc. In certain embodiments, the device is attach it to the handlebars so that it can easily access while riding.

The pressure sensors are attached in locations on the cycle to measure rider physical inputs. This can be done by using a flexible adhesive or small puck to attach the sensors to a seat, saddle, bicycle peddle, motorcycle peg, horse stirrup or handlebars, etc. These locations can be protected from damage and can simultaneously measure rider physical inputs, which is unlike anything previously utilized.

In this and other embodiments, motion tracking is a process used to measure and analyze the physical movements of an individual or object. It is a useful tool for many applications, such as biomechanics, sports performance, and rehabilitation. To more accurately track motion, a flexible adhesive or small puck can be attached to a seat, saddle, bicycle peddle, motorcycle peg, horse stirrup, or handlebars, etc. This allows for the unique measurement of rider physical inputs, while also protecting the locations from damage.

The adhesive or puck can be chosen based on the type of motion being tracked. For example, a flexible adhesive may be more suitable for tracking movements on a seat or saddle where such a thin strip would be non-invasive, such as those associated with cycling, while a more rigid puck may be better suited for locations such as the pedals of a bicycle, the pegs of a motorcycle or the stirrups of a horse or the frame rails of a bicycle or motorcycle.

With the adhesive or puck is in place, it can be connected to a tracking device. This device can be used to measure and analyze the rider's physical inputs, such as force. The data collected can then be used to create a detailed analysis of the rider's performance. This information can be used to identify areas for improvement, as well as to provide feedback on the rider's progress.

In addition to the motion tracking device, other components can be utilized to ensure accurate and reliable results. For example, a power source is typically used to power the device and any data storage system used to store the data collected. Furthermore, a software program may be needed to interpret the data and generate reports.

In this and other embodiments, motion tracking is a powerful tool for measuring and analyzing physical inputs. By attaching a flexible adhesive or small puck to a seat, saddle, bicycle peddle, motorcycle peg, horse stirrup, or handlebars, etc., a rider (or other user) can be accurately tracked, while also protecting the locations from damage. The data collected can then be used to create detailed analyses of rider performance, allowing for the identification of areas for improvement and providing feedback on progress.

In this and other embodiments, the current disclosure teaches, the device does not need to for the saddle or seat to be modified or to affect the saddle or seat. Rather the device merely senses whether the rider is activating the saddle or the seat. This is advantageous in the device can then be later removed without changing the cycle (and its saddle and seat) so that muscle memory of the riding (and what has be learned by the tracking of the device) will not have to change due to alterations of the cycle.

By using such embodiment, the rider can track their activities and view a map chart of where the rider's physical inputs on the bike/horse/motorycle or other vehicle have been suboptimal based on the GPS data from my CPU. I.e., the rider is able to see how the rider performed while rider the cycle, and see when (and where) the rider should have rider the cycle differently.

For example, a road-rider can use the device to track the power delivery from the rider's legs to each pedal of the cycle to ensure equal power delivery. The rider can attach the pressure sensor to each pedal and then go for a ride. After the ride, the rider can open an app on the rider's cellphone (or other computer device) and is able to view a map chart of the trail the rider rode along with data from the device CPU which informs the rider where the rider's pressure was even and consistent and where it was not.

By such process, embodiments of the present invention teach a method of displaying data from a GPS receiver, a CPU, and a wireless transmitter. This can be done by using a combination of hardware and software. The software can include an app or web application that can receive and process the data from the CPU and GPS receiver. The app or web application would then be able to chart and display the rider's activities along with a map chart of where the activity occurred based on the GPS data. The app or web application would also be able to store the data for future use.

Additionally, each of the data streams from each of the sensors can be displayed individually or collectively with other data streams, in a user chosen variety of colors to distinguish each data stream.

Furthermore, motion tracking is a process that can combine both hardware and software components to track and record a rider's movements. This process can be used for various applications such as fitness tracking, navigation, and surveillance. The hardware components of motion tracking include a GPS receiver, a CPU, and a wireless transmitter. The software component can again be an app or web application that can receive and process data from the CPU and GPS receiver.

The app or web application is capable of charting and displaying a rider's activities along with a map chart of where the activity occurred based on the GPS data. This allows the rider (other user) to view their activity in relation to their location. The app or web application is also capable of storing the data for future use. This allows the rider to review their activity over time and make changes to their routine if needed.

Each of the data streams from each of the sensors can be displayed individually or collectively with other data streams, in a user chosen variety of colors to distinguish each data stream. This allows the rider to easily identify and interpret the data streams. The data streams can also be used to create a comprehensive report of the activity. This report can be used to analyze the activity and make changes to the routine if needed.

The force and motion tracking of the device can include both hardware and software components to track and record a rider's movements. The hardware components can include a GPS receiver, a CPU, and a wireless transmitter. The software component can include an app or web application that can receive and process data from the CPU and GPS receiver. The app or web application is able to chart and display a rider's activities along with a map chart of where the activity occurred based on the GPS data. It is also able to store the data for future use. Each of the data streams from each of the sensors can be displayed individually or collectively with other data streams, in a user chosen variety of colors to distinguish each data stream. This allows the user to easily identify and interpret the data streams.

### Cycling System

Representative of the present invention is a device used by a cyclist or motorcyclist.

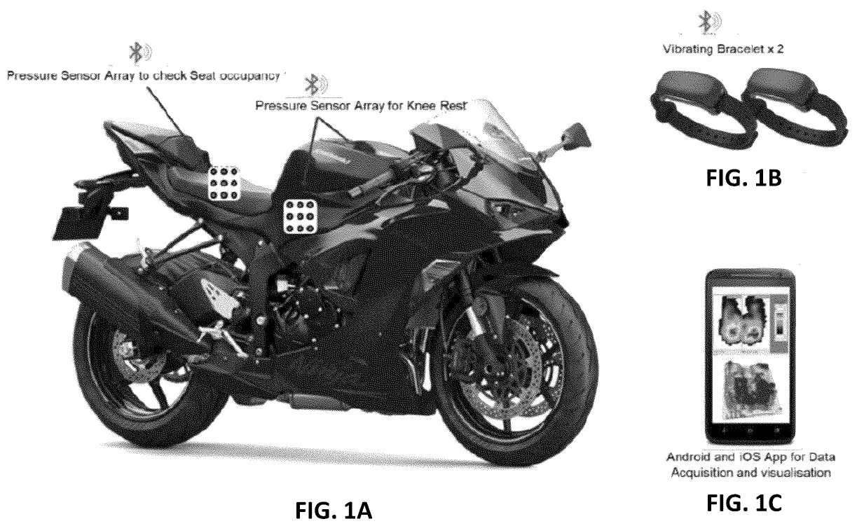

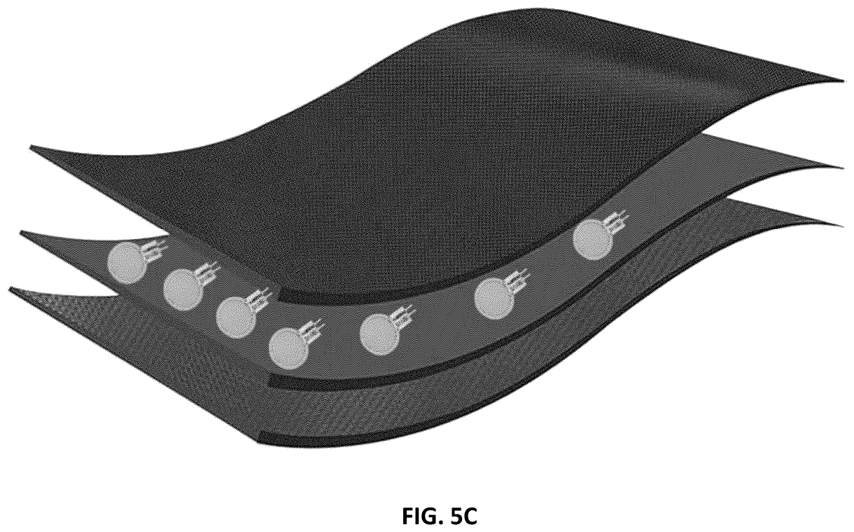

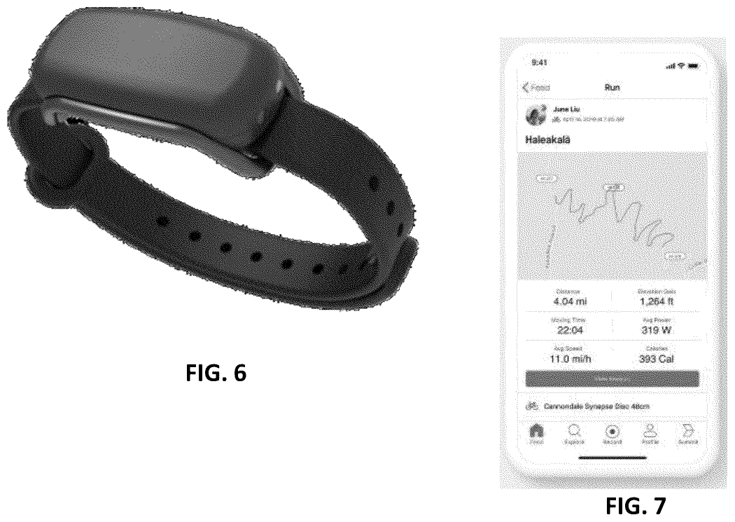

FIGS. 1A-1C are illustrations showing a system of the present invention. FIG. 1A shows a motorcycle that includes a pressure sensor array for the seat of the motor cycle, and pressure sensor arrays for each of the left and right knee rests. Such sensor arrays can be in the form of a fabric, which can be fixed on a seat or on the rider's knee rests with a flexible attachable mechanism (such as a Velco material). FIG. 1B shows vibrating bracelets that can be worn on the left and right wrist of the rider. See also FIG. 6. Such bracelets can vibrate to indicate incorrect seating position. FIG. 1C shows a mobile device that can provide visual data via a mobile app. See also FIG. 7. The mobile app can also collect the left, right, and seat sensor data and display as a graph for analytic purposes. Each of these can work on blue-tooth technology (i.e., blue-tooth emitters) and can be battery operated.

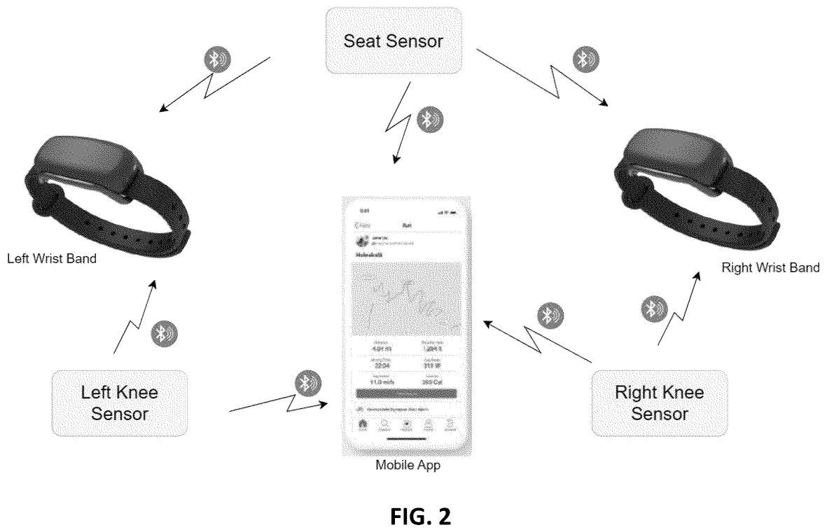

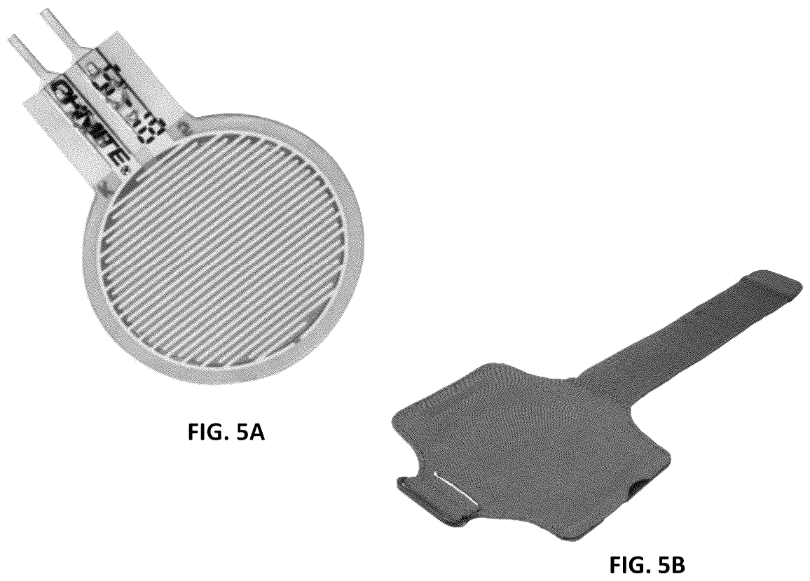

For example, the left knee, right knee, and seat sensors read pressure data from array of 4×4 sensors mechanically arranged so that it can sense pressures up to 16 positions. As shown in FIG. 2, the left/right knee/seat sensor data is transmitted over BLE beacon, which is collected by the left/right wrist band. FIG. 5C is an exploded view of the flexible pressure array (shown in FIG. 5B), which includes a top layer (fabric), a bottom layer (felt), and a middle layer (flexible PCB with the array of pressure sensors, as shown in FIG. 5A).

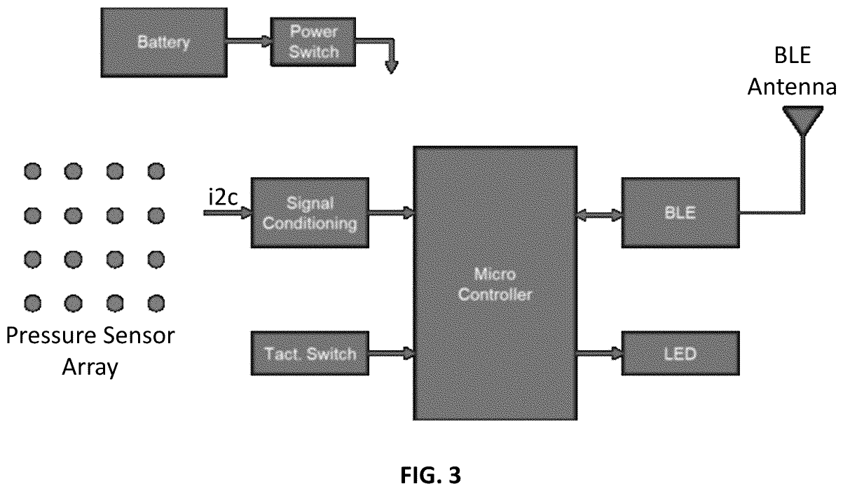

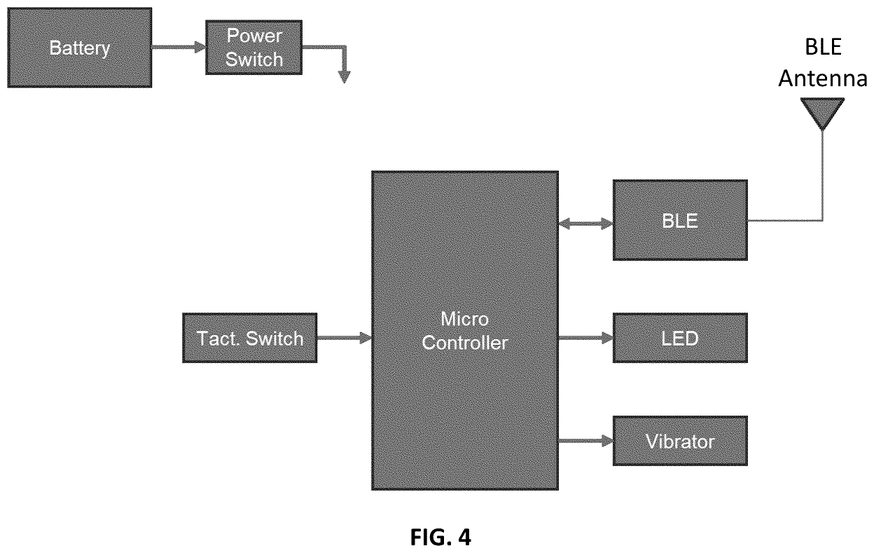

FIG. 3 shows a block diagram for the sensor array for the seat and knee rest. FIG. 4 shows a block diagram for vibrating bracelet that can be used in the embodiment shown in FIG. 1B.

Such system as shown in FIG. 1A-1C include a number of transmission devices (currently based on pressure data) in the form of a flexible fabric that can be moved on the body, as well as two receiving devices—in the form of armbands that transmit haptic feedback from any two of the sensors, with the user having the ability to determine which sensor will provide the feedback to the armband.

The sensors will also transmit data to an application-both android and IOS which will track and record all data streams, via color code/over GPS map etc.

Such system can be modified and or be versatile in a number of use cases, including as follows:
Transmissions from the left side sensors can be sent to the left side band.
Transmissions from the right-side sensor should be sent to the right-side band.
Transmissions from the seat sensors can be sent simultaneously to both bands, with a different type of feedback to distinguish from left or right sensor feedback.
If there is an accelerometer is on the seat, via a fabric, the rider should have the ability to elect to disable the feedback from the device while it still being recorded in the app.
The bands (and the app) should be able to record data from a variety of sources such as pressure plates and gyroscope devices-such as a helmet-based gyroscope sensor that will determine whether a user is riding while looking ahead (level) or whether the user is looking down (not level).
There can be similar gyroscopic sensors that will end up near the elbow so the rider can determine if the rider is riding with his elbows up (what is known as the attack position) or whether the rider is riding passively with the rider's elbows down.
The target number of sensors that need to be tracked could include-foot-based sensors, an ankle of lower leg based sensor, and a thigh based sensor. There could be a seat-based sensor and on the upper body it could be elbow based and helmet based.
One aspect is that the app can record, track, and convey all data while the rider can be able to select one of the data transmissions to trigger the left band, right band, or simultaneously trigger both bands.
In embodiments, the rider can be able to program the level of sensor sensitivity which will trigger the sensor as being “activated” vs “non-activated”-subject to a floor and a ceiling.
The rider can be able to select the level of intensity of the armband signal-subject to a ceiling.
The rider can also be able to select which sensor he wants to transmit to the receiver.
The system can be able to simultaneously accommodate date from the foot, ankle, lower thigh (above the knee) and upper thigh, provide seated data and be able to record gyroscopic data from the head (attached to the helmet) and arms (a band directly above or below the elbow. That leads to a possible twelve simultaneous sensor readings, transmission, and recording (or more).
As far as triggering the sensors, all left side sensors can have the ability to trigger the left side armband (and a rider can be be able to designate which armband/sensors are L/R.
The seat sensor and helmet sensor can trigger both armbands simultaneously. These can be done with different pulses so a rider can track both at the same time. In embodiments that there are not different pulses to the armband, the rider can utilize the seat and/or helmet sensor.

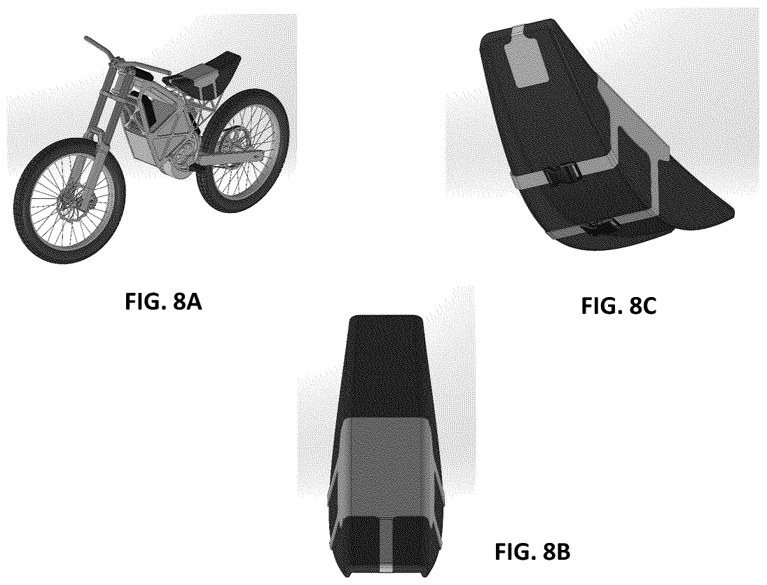

FIGS. 8A-8C shows a motorcycle with a seat pressure sensor that can be utilized. As shown in FIG. 8A, the motorcycle has a seat pressure sensor that has been attached. FIG. 8B shows the seat from overhead with the seat pressure sensor. The array of pressure sensors in this array face upward and are position to contact the rider when the motorcycle is ridden. FIG. 8C a shows the underside view of the seat and that the pressure sensor is attached (by straps and a buckle).

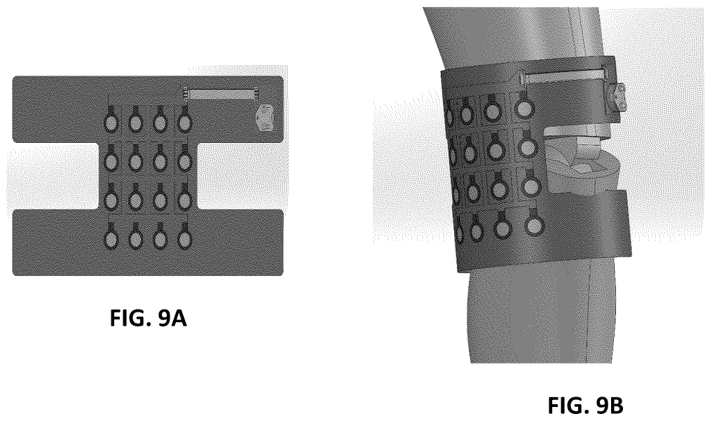

FIGS. 9A-9B shows knee sensors that are attached to the rider (as opposed to being positioned on the motorcycle as shown in FIG. 1A. FIG. 9A shows the flexible sensor array of pressure sensors that can be attached to a knee of the rider. This includes a transmitter (such as a blue-tooth transmitter) for transmission of the information sensed by the array of pressure sensors. FIG. 9B shows the flexible sensor array when attached to a knee of the rider. Note that the array of pressure sensors would be positioned on the in-seam of the rider, such that the pressure sensors would come in contact with the motorcycle (or other movable object), when ridden by the rider.

### Further Variations

A number of further variations can be implements in embodiments of the present invention.

There can be a manual way to disable each of the wearables-seat, left leg, and right leg. The disable feature could, for example, disable transmission to the respective arm band but not to the app on the phone. Accordingly, if a rider wanted to work on the rider's left hand cornering, the impact of the right leg pressure sensor is relevant (whereas the impact of the left leg pressure sensor is less relevant). By the same token, there are many situations where sitting on the seat is preferable to standing so that signal would need to be disabled to the armband.

The system can include a feature of auto-stopping (to the band and/or the app) when there is no movement. (e.g., the cycle is stopped). Alternatively or additionally, if a signal can be sent to the app indicating the cycle is stopped (not in motion), this can enable the rider to review the data during the stopped time period. (For safety reasons, this can be precluded during movement of the cycle).

The app can track all sensors independently so a rider can, for example, view left leg, right leg and seat data independently. This can be overlayed over a GPS map so the rider can see where and how the rider performed on each section of the track or trail.

With respect to the physical sensors on the rider, the surface area available may be limited, so that the pressure sensors need to be adjusted. For instance, grip areas can have limited surface areas. Accordingly, there should be a user adjustable minimum amount of pressure necessary so that the “no contact” signal is not sent. The idea is not for a rider merely to have minimum contact with the bike but to control it with the rider's lower body—for that there needs to be pressure. The rider can be able to dial in what that minimum threshold is.

The pressure sensors strip can be flexible enough that it can “wrap” around the frame of the cycle or other movable object depending on how the rider grips the bike. In embodiments, a rider will not have contact with all pressure points on the sensor, (and the rider will make contact with only a fraction of the pressure points). The device can record the number of pressure points making contact and that the aggregate overall pressure of those particular contact points is enough to meet the minimum pressure threshold the rider has selected. On the app though, the rider would still be able to track how many of the contact patches were activated.

The armband can have user adjustable levels of intensity with respect to the delivery of the vibration/shock to the arm. These can range in intensity. However, a light level of pressure may not be enough signal to a rider in a stressful or intense training situation.

While embodiments of the invention have been shown and described, modifications thereof can be made by one skilled in the art without departing from the spirit and teachings of the invention. The embodiments described and the examples provided herein are exemplary only, and are not intended to be limiting. Many variations and modifications of the invention disclosed herein are possible and are within the scope of the invention. The scope of protection is not limited by the description set out above, but is only limited by the claims which follow, that scope including all equivalents of the subject matter of the claims.

The disclosures of all patents, patent applications, and publications cited herein are hereby incorporated herein by reference in their entirety, to the extent that they provide exemplary, procedural, or other details supplementary to those set forth herein.

Amounts and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a numerical range of approximately 1 to approximately 4.5 should be interpreted to include not only the explicitly recited limits of 1 to approximately 4.5, but also to include individual numerals such as 2, 3, 4, and sub-ranges such as 1 to 3, 2 to 4, etc. The same principle applies to ranges reciting only one numerical value, such as “less than approximately 4.5,” which should be interpreted to include all of the above-recited values and ranges. Further, such an interpretation should apply regardless of the breadth of the range or the characteristic being described.

Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are now described. Following long-standing patent law convention, the terms “a” and “an” mean “one or more” when used in this application, including the claims.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that can vary depending upon the desired properties sought to be obtained by the presently disclosed subject matter.

As used herein, the term “about” and “substantially” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, the term “substantially perpendicular” and “substantially parallel” is meant to encompass variations of in some embodiments within ±10° of the perpendicular and parallel directions, respectively, in some embodiments within ±5° of the perpendicular and parallel directions, respectively, in some embodiments within ±1° of the perpendicular and parallel directions, respectively, and in some embodiments within ±0.5° of the perpendicular and parallel directions, respectively.

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

### REFERENCES

CN-201880027571A: Seat type sports simulation amusement park scenery spot (CN—Apr. 23, 2018): [Universal City Studios LLC].
GB-2016051363-W: Saddle (GB—May 12, 2016): [Ergon Equine Ltd; Ergon Equine Limited].
EP-16723469-A: Saddle (Ep—May 12, 2016): [Ergon Equine Ltd].
US201615573036-A: Saddle (US—May 12, 2016): [Ergon Equine Ltd].
EP-10700273-A: Saddle (EP—Jan. 6, 2010): [Brander Andrew Michael; Brander Donna Kaye; Ward Tracey Elizabeth; and Ward Mathew Roger].
US201013142646-A: Saddle (US—Jan. 6, 2010): [Brander Andrew Michael; Brander Donna Kaye; Ward Tracey Elizabeth; and Ward Mathew Roger].
US201514965774-A: Bicycle Saddle (US—Dec. 10, 2015): [Dudley II Jackson C.].

What is claimed is:

1. A system for motion tracking, capturing, and transmitting a rider contact and input with a movable object, wherein the system comprises:
(a) one or more pressure sensors that are each removably attachable to the movable object or rider such that the one or more pressure sensor can be placed at one or more locations of the movable object or rider, wherein the one or more pressure sensors are located on the movable object or rider to detect the presence and amount of force applied by the rider to the movable object at the one or more locations;
(b) a tracker removably attachable to the movable object or the rider, wherein the tracker is capable of tracking where the movable object is;
(c) a recorder that is operably connected to the one or more pressure devices and the tracker, and wherein the recorder is operable to record information comprising:
(i) the presence and amount of force applied by the rider to the movable object at the one or more locations, and
(ii) where the movable object is at the time when the presence and amount of force applied by the rider to the movable object at the one or more locations; and
(d) an indicator that is operable to provide real-time feedback to the rider related to the information recorded by the recorder, wherein the indictor is removably attachable to the movable object or the rider.

2. The system of claim 1, wherein the one or more pressure sensors are wirelessly connected to recorder.

3. The system of any of claim 1, wherein the recorder comprises a CPU that is operable to record times of contact and times of non-contact of the one or more pressure devices.

4. The system of claim 3, wherein
(a) the tracker comprises a GPS system; and
(b) the CPU is operable to transmit the information from the one or more pressure sensors and the GPS system to a mobile device.

5. The system of claim 1, wherein the tracker comprises a GPS system.

6. The system of claim 1, wherein the tracker comprises an accelerometer.

7. The system of claim 6, wherein the accelerometer tracks tilt of the movable object.

8. The system of claim 1, wherein
(a) the system comprises a combination of hardware and software,
(b) the hardware comprises a GPS receiver, a CPU, and a wireless transmitter, and
(c) the software comprises an app or web application that can receive and process data from the CPU and the GPS receiver, wherein
(i) the app or web application is operable to chart and display the rider's activities along with a map chart of where the activity occurred based on the GPS data; and
(ii) the app or web application is operable to store the data for future use.

9. The system of claim 1, wherein the recorder is operable for downloading information recorded by the recorder to a storage device that is not attached to the movable object or the rider.

10. The system of claim 1, wherein at least one of the one more pressure sensors is removably attachable to a part of the movable object selected from the group consisting of a seat, saddle, bicycle peddle, motorcycle peg, horse stirrup, and handlebar of the movable object.

11. The system of claim 1, wherein the indicator provides feedback to the rider that is visual response.

12. The system of claim 1, wherein the indicator provides feedback to the rider that is an audible response.

13. The system of claim 1, wherein the system provides feedback to the rider that is a tactile response.

14. The system of claim 1, wherein
(a) the indicator comprises a plurality of wearable indicators;
(b) the plurality of wearable indicators comprise a right-side wearable indicator and a left-side wearable indicator;
(c) the right-side wearable indicator is removably attachable to a right wrist or a right arm of the rider; and
(d) the left-side wearable indicator is removably attachable to a left wrist or a left arm of the rider.

15. The system of claim 14, wherein the right-side wearable indicator and the left-side wearable indicator are independently controllable.

16. The system of claim 1, wherein:
(a) the one or more pressure sensor comprises a first pressure sensor, a second pressure sensor, and a third pressure sensor, wherein
(i) the first pressure sensor is removable attachable at a first location that is at a right knee rest on the movable object or at or by a right knee of the rider,
(ii) the second pressure sensor is removable attachable at a second location that is at a left knee rest on the moveable object or at or by a left knee of the rider, and
(iii) the third pressure sensor is removable attachable to a seat of the movable object; and
(b) the indicator comprises a first wearable indicator and a second wearable indicator, wherein
(i) the first wearable indicator is removably attachable to a right wrist or a right arm of the rider; and
(ii) the second wearable indicator is removably attachable to a left wrist or a left arm of the rider.

17. The system of claim 16, wherein the system is operable in real time to:
(a) provide a first set of indications to the rider, via the first wearable indicator, based upon information recorded from the recorder obtained from the first pressure sensor;
(b) provide a second set of indications to the rider, via the second wearable indicator, based upon information recorded from the recorder obtained from the second pressure sensor; and
(c) provide a third set of indications to the rider, simultaneously via the first wearable indicator and the second wearable indicator, based upon information recorded from the recorder obtained from the third pressure sensor.

18. A method for motion tracking, capturing, and transmitting a rider contact and input with a movable object, wherein the method comprises:
(a) selecting a system that comprises
(i) one or more pressure sensors that are each removably attachable to the movable object or rider,
(ii) a tracker removably attachable to the movable object or the rider,
(iii) a recorder that is operably connected to the one or more pressure devices and the tracker, and
(iv) an indicator removably attachable to the movable object or the rider;
(b) attaching the one or more pressure sensors to the movable object or rider such that the one or more pressure sensor are placed at one or more locations of the movable object or rider;
(c) attaching the tracker to the movable object or the rider; and
(d) utilizing the system when the rider is riding the movable object, wherein utilizing the system comprises
(i) the one or more pressure sensors detect the presence and amount of force applied by the rider to the movable object at the one or more locations,
(ii) the tracker tracks where the movable object is,
(iii) the recorder records information comprising
(A) the presence and amount of force applied by the rider to the movable object at the one or more locations, and
(B) where the movable object is at the time when the presence and amount of force applied by the rider to the movable object at the one or more locations, and
(iv) the indicator provides real-time feedback to the rider related to the information recorded by the recorder.

19. The method of claim 18, wherein the method further comprises the rider receives real-time feedback from the indicator while riding the movable object.

20. The method of claim 18, wherein,
(a) the system is the system of claim 19;
(b) when the rider is riding the movable object outside pre-set parameters for the position of a right knee of the rider relative to a right knee rest of the movable object, the first wearable indicator provides an indication to the rider;
(c) when the rider is riding the movable object outside pre-set parameters for the position of the left knee of the rider relative to a left knee rest of the movable object, the second wearable indicator provides an indication to the rider; and
(d) when the rider is riding the movable object outside pre-set parameters for the position of the rider relative to seat of the movable object, the first wearable indicator and the second wearable indictor simultaneously provide an indication to the rider.
